(19) United States
(12) Patent Application Publication
(10) Pub. No.: US 2008/0284845 A1
(43) Pub. Date: Nov. 20, 2008

(54) METHOD AND APPARATUS TO PROCESS ENDOSCOPE IMAGES

(75) Inventors: Til Aach, Aachen (DE); Thomas Stehle, Aachen (DE)

Correspondence Address: RANKIN, HILL & CLARK LLP, 38210 Glenn Avenue, WILLOUGHBY, OH, 44094-7808, US

(73) Assignee: OLYMPUS WINTER & IBE GMBH, Hamburg (DE)

(21) Appl. No.: 12/120,446

(22) Filed: May 14, 2008

(30) Foreign Application Priority Data
May 14, 2007 (DE) 10 2007 022 888.2

(51) Int. Cl. A61B 1/04; H04N 7/18

(52) U.S. Cl. 348/71; 348/E07.087

(57) ABSTRACT

The invention relates to a method improving the images in an image processing unit (5) receiving images from an electronic color video camera (3) of a medical endoscope (1) and then transmitting them to an image display unit (7), and to an endoscope operating on the basis of the said method, where first the pixels of the color components (RGB) are individually mapped into a color space (HSL) wherein the color saturation (S) is independent of the other components (H, L), thereupon the saturation component (S) of each pixel to be mapped is converted by means of a nonlinear mapping function (11) which amplifies the color saturation differential between an upper zone (b->1) and a lower zone (0-b) of the color saturation, lastly the pixels being remapped into an (RGB) color space suitable for image display.

### BACKGROUND OF THE INVENTION

Image processing is used in medical endoscopy to gain better insight into definite images.

Known image-processing methods and apparatus are used in medical endoscopy for instance to accentuate given colors, to emphasize the organ structures in weakly structured images, and the like. Conventional image processing procedures are used for such purposes as raising the contrast, color changes and the like.

Whitish tissue structures raise a problem which to-date has not been overcome satisfactorily. Such tissue structures in particular are fasciae, that is thin, transparent skins and also nerves. Such tissues exhibit hardly any color of their own, are translucent and look whitish or milky. The colors of organs underneath them glimmer though them with attenuated intensity.

Whitish tissue structures such as fasciae or nerves—usually situated above organs displaying strongly colored organs, usually can be detected only with difficulty. Their detectability is improved only inadequately by conventional image processing techniques.

The objective of the present invention accordingly is to improve the visual perception of whitish, milky tissue structures using image processing means.

### BRIEF SUMMARY OF THE INVENTION

The present invention is based on the insight that the perception of a whitish, milky and translucent image is best described not by the lightness of color hues, but by color saturation. The milky, whitish perception therefore can be changed by altering the color saturation. Preferably the remaining perception should remain unchanged. Consequently the present invention calls for mapping the color values during pixel-wise image processing, namely the pixel color components generated by the video camera in a given color space—for instance the conventional RGB color space (red, blue, green)—are first transformed into a color space wherein the color saturation is independent of the other components. Illustratively the HSL (Hue, Saturation, Lightness) color space is appropriate, or the HSV (Hue, Saturation, Value) color space. In this new color space wherein saturation is an independent component processed relative to the other components (H, L), the color saturation is mapped pixelwise as a nonlinear function from an input into an output signal, said function being such as to reinforce the color saturation differential between a zone of higher and a zone of lower color saturation. Thereupon, and pixel-wise, the remapping is performed into a color space suitable for image display. Typically this shall be again the RGB color space. The image processing of the present invention alters the color saturation, the other color components, namely the hue or the lightness, remaining unchanged. The color saturation being applied to the whitish, milky appearance of the structures to be emphasized, they may thereby be emphasized relative to the surrounding zones. Only color saturation being affected, not the hue or the lightness, the perception of other image details remains substantially as before in this kind of image processing. Nerves—which inherently are difficult to scrutinize—also can be made distinct. Only color saturation being affected, not hue or lightness, such image processing will preserve the other image details' substantially unaltered appearance.

When mapping the color saturation, the mapping function (also “characteristic line”) may be selected in a manner that the zones of higher saturation shall be amplified in order to raise the color saturation contrast compared to the less saturated fasciae or nerves. Preferably however, such a mapping function shall be selected that the color saturation shall be further attenuated in the zones of lesser color saturation. This feature reinforces for instance the fasciae's milky appearance, while strongly colored other organs keep their appearance unchanged. In this manner the image will appear more natural.

A mapping function shall be used which within the attenuating zone shall attenuate higher color saturations more than at lesser color saturations. In this manner and as illustratively regards displaying a fascia of variable thickness, the thinner and thicker zones are optically made to appear, by image processing, more like each other, the fasciae thereby being equally visible everywhere.

The mapping function is advantageously adjustable to values even applicable to the entire image. Illustratively a mapping function well suited to display certain fasciae may be selected, or a mapping function especially appropriate to display nerves. Also the mapping function may be switched to “Identity”, in order to entirely eliminate color saturation control effects, to allow examining of the natural image.

On the other hand, depending on given parameters, different mapping functions also may be used from pixel to pixel. Illustratively, the mapping function can be adjusted depending on the lightness and/or the hue of the particular pixel. In the case of lightness dependence, a linear mapping function may be used for very dark image sites in order to avert changes in color saturation in such dark zones and to preserve to the extent possible all still available image perceptions. In the case of hue dependence, given colors wherein color saturation remains remain constant and illustratively not arising in fasciae may be suppressed again in order to maintain the natural image perception.

Advantageously, the mapping function may be adjusted based on the image structural data. The overall image, wherein structures such as organ edges were ascertained by other image processing means, then must be taken into account at each pixel mapping. These means allow detecting, for instance, a fascia's edge and to emphasize it in especially marked manner relative to the adjoining tissue by controlling the color saturation.

Preferably, statistical data from the overall image are included to control the mapping function. As known from image processing manuals, histogram computations are especially well suited for such purposes. Illustratively conventional histogram spreading or histogram equalization may be used in image processing. In this manner the color saturations may be optimally distributed in the available range of values.

The present invention is illustratively and schematically shown in the appended drawings.

### BRIEF DESCRIPTION OF THE DRAWINGS

FIG. 1 is a functional view of an endoscope with an image processing unit and an image display unit,

FIG. 2 is an enlarged schematic view of the image processing unit,

FIG. 3 is a plot of the mapping function used, and

FIG. 4 shows a section of a body organ with two superimposed fasciae.

### DETAILED DESCRIPTION OF THE INVENTION

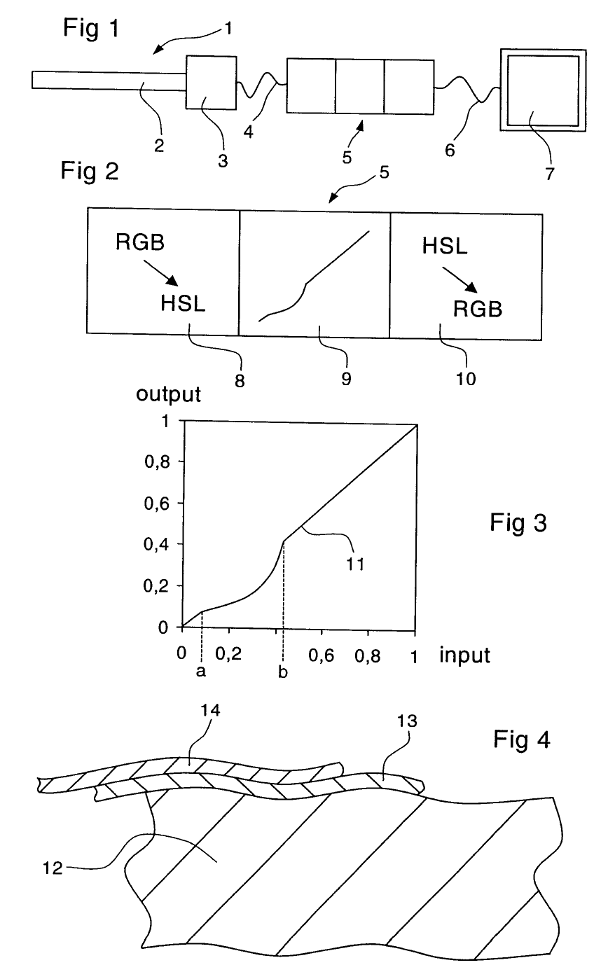

FIG. 1 shows a medical endoscope 1 fitted with an elongated stem 2, a color video camera 3 being mounted at said stem's proximal end. In another design, the camera 3 also may be configured in the distal end zone of the stem 2 directly behind the camera lens.

The color video camera 2 is connected to a cable 4 used both for data transmission and illustratively also for electric power, said cable being connected to an image processing unit 5 to which it feeds image data. By means of a cable 6, the image processing unit 5 is connected to an image display unit 7, for instance a conventional monitor.

Illustratively, the endoscope 1 may be used for laparoscopy and in that case it shall be inserted by its stem 2 through a lancing aperture into the belly area to view organs situated therein. The image seen by the color video camera 3 is recorded and transferred to the image processing unit 5 where it is processed and then displayed on the image display unit 7.

FIG. 2 shows the image processing unit 5 in detail. It comprises three stages 8, 9, 10 wherein the image pixels are processed consecutively.

In the first stage 8, the pixels—from the RGB color space used in this embodiment mode by the color video camera 3, or from another color space used in the camera—are individually mapped into another color space wherein the saturation S is independent from the remaining color components. In this illustrative embodiment mode, the HLS (Hue, Saturation, Lightness) color space is used. However the typically used HSV (where V is value) may also be used.

In the second image processing stage 9, the color saturation S is mapped using a mapping function 11 in the HSL color space. Next, in the third stage 10, the HSL signal with the mapped saturation values is remapped into the RGB space which is appropriate in this embodiment mode for display on the image display unit 7. Mapping also may take place into another color space that would be more appropriate for another image display unit.

Also, otherwise than shown in FIG. 1, the image processing unit 5 may be integrated into the endoscope 1 or the image display unit 7.

FIG. 3 shows the mapping function 11 as a plot of ordinate output values depending on the abscissa input values, the ordinate and the abscissa each ranging from 0 to 1.

The mapping function 11, illustratively shown in FIG. 3, is discussed below. The dots on the curve 11 denote the abscissa values corresponding to their input color saturations.

The mapping function 11 includes two zones on the rising diagonal curve between the values 0 and a and the values between b and 1. Within these zones, the color saturation is transmitted identically. Within the zone a->b, the mapping function deviates from “Identity” in a manner that attenuation results. Illustratively FIG. 3 shows that the input color saturation of 0.2 results in an output saturation of 0.1.

FIG. 4 shows a typical application. It represents a section of a body organ 12, for instance an intestine, liver or the like. In the present hypothetical, didactic case, two fasciae 13, 14 rest on the organ 12, and, as indicated in FIG. 4, they overlap differentially the organ 12. When the endoscope 1 looks from above on the organ 12, it will see the organ proper to the right in FIG. 4, then at the middle the organ covered only by the fascia 13 and then at the left part of the organ 12 the same being covered by both fasciae 13 and 14.

In most cases organs such as 12 are strongly colored, for instance being reddish, brownish or the like. The fasciae 13, 14 are colorless, translucent in a milky way, as a result of which, where covered by the fasciae, the organ color still shows, though at a lesser saturation.

When using color saturation mapping using the mapping function 11 of FIG. 3, no change will take place in the upper color saturation zone b->1 of the mapping function 11. Accordingly, the strongly colored organ 12 is shown unchanged where its surface is bared. In that zone where the organ 12 is covered only by the fascia 13, the color saturation is attenuated because being less than b. The color saturation is attenuated and as a result the milky perception of the fascia 13 is reinforced. Where the two fasciae 13, 14 are superposed, the color saturation is especially low. Near the point a, the color saturation attenuation becomes less. In the zone of overlap of the two fasciae 13 and 14, therefore the color saturation is less than where there is only one color. The full region containing any fascia therefore is rendered uniformly in its milky appearance. Differences within the fasciae's region are less. This feature is especially valuable for fasciae of different thicknesses and less perceptible in some places than others. They are shown having substantially the same thicknesses, as a result of which they may be properly recognized also where they are thin.

Slight attenuation also may take place in the range 0—a of the mapping function 11. In the shown embodiment mode of the mapping function 11, however, no further lowering of the already low color saturation will take place because it would not result in further image improvement.

The mapping function 11 may be varied from that shown in FIG. 3, for instance where it is desirable to emphasize the zones of different thicknesses within fascia-covered zones.

The mapping function 11 illustratively also may be switched to “Identity” and in that case it will consist of a rising diagonal. In such a case, the variation of color saturation is entirely shut off and the natural image can be viewed.

Illustratively, as shown in FIG. 3, the mapping function 11 may be maintained for all pixels. However it also may be compared pixel after pixel, for instance using the other pixel color components H or L. Said function also may be adjusted using data from the pixel's vicinity, for instance structure data, or for instance using statistical data based on the entire image.

1. A method for improving images of an image processing unit that receives images from an electronic color video camera of a medical endoscope to transfer them to an image display unit,
wherein pixels of color components of a color space used by the video camera are transformed individually into a color space in which color saturation is independent of other components, wherein next the color saturation of each pixel to be mapped is processed by a non-linear mapping function that amplifies the color saturation between an upper zone and a lower zone of the color saturation, and wherein, lastly, the pixels are remapped into color space suitable for image display.

2. The method as claimed in claim 1, wherein the mapping function attenuates the color saturation in a zone of lesser color saturation.

3. The method as claimed in claim 2, wherein, within the color saturation attenuating zone, higher color saturations are more attenuated than lower ones.

4. The method as claimed in claim 1, wherein the mapping function is adjustable.

5. The method as claimed in claim 4, wherein the mapping function is adjusted based on one of lightness and hue of the particular pixel.

6. The method as claimed in claim 4, wherein the mapping function is adjusted based on image structure data.

7. The method as claimed in claim 4, wherein the mapping function is adjusted based on the image's statistical data.

8. A medical endoscope fitted with a color video camera, an image processing unit connected to said camera, and an image display unit connected to said processing unit,
wherein the image processing unit (5) is designed to implement the method defined in claim 1.
